(19) United States
(12) Patent Application Publication
(10) Pub. No.: US 2007/0179396 A1
(43) Pub. Date: Aug. 2, 2007

(54) Method and System for Detecting and Classifying Facial Muscle Movements

(75) Inventors: Tan Thi Thai Le, New South Wales (AU); Nam Hoai Do, New South Wales (AU); Marco Kenneth Della Torre, New South Wales (AU); William Andrew King, New South Wales (AU); Hai Ha Pham, New South Wales (AU); Emir Delic, New South Wales (AU); Johnson Thie, New South Wales (AU); Briony Marie Doyle, New South Wales (AU); Vivian Lo, New South Wales (AU)

Correspondence Address: FISH & RICHARDSON P.C., PO BOX 1022, MINNEAPOLIS, MN, 55440-1022, US

(73) Assignee: EMOTIV SYSTEMS PTY LTD, New South Wales (AU)

(21) Appl. No.: 11/531,117

(22) Filed: Sep. 12, 2006

Related U.S. Application Data
(63) Continuation-in-part of application No. 11/225,598, filed on Sep. 12, 2005.

(51) Int. Cl. A61B 5/04; A61B 5/103

(52) U.S. Cl. 600/544; 600/595

(57) ABSTRACT

A method of detecting and classifying facial muscle movements, comprising the steps of: receiving bio-signals from at least one bio-signal detector; and applying at least one facial muscle movement-detection algorithm to a portion of the bio-signals affected by a predefined type of facial muscle movement in order to detect facial muscle movements of that predefined type.

### CROSS-REFERENCE TO RELATED APPLICATIONS

This application is a continuation-in-part of and claims priority to U.S. application Ser. No. 11/225,598, filed on Sep. 12, 2005, which is incorporated by reference.

### FIELD

The present invention relates generally to the detection and classification of facial muscle movements, such as facial expressions or other types of muscle activity, in human subjects. The invention is suitable for use in electronic entertainment or other platforms in which electroencephalograph (EEG) data is collected and analysed in order to determine a subject's facial expression in order to provide control signals to that platform, and it will be convenient to describe the invention in relation to that exemplary, non-limiting application.

### BACKGROUND

Facial expression has long been one of the most important aspects of human to human communication. Humans have become accustomed to consciously and unconsciously showing our feelings and attitudes using facial expressions. Furthermore, we have become highly skilled at reading and interpreting facial expressions of others. Facial expressions form a very powerful part of our everyday life, everyday communications and interactions.

As technology progresses, more of our communication is mediated by machines. People now “congregate” in virtual chat rooms to discuss issues with other people. Text messaging is becoming more popular, resulting in new orthographic systems being developed in order to cope with this unhuman world. Currently, facial expressions have not been used in man machine communication interfaces. Interactions with machines are restricted to the use of cumbersome input devices such as keyboards and joysticks. This limits our communication to only premeditated and conscious actions.

There therefore exists a need to provide technology that simplifies man-machine communications. It would moreover be desirable for this technology to be robust, powerful and adaptable to a number of platforms and environments. It would also be desirable for this technology to optimise the use of natural human to human interaction techniques so that the man-machine interface is as natural as possible for a human user.

### SUMMARY

With this in mind, one aspect of the invention provides a method of detecting and classifying facial muscle movements, including the steps of receiving bio-signals from at least one bio-signal detector, and applying at least one facial muscle movement-detection algorithm to a portion of the bio-signals affected by a predefined type of facial muscle movement in order to detect facial muscle movements of that predefined type.

The step of applying at least one facial movement-detection algorithm to the bio-signals may include:

comparing the bio-signal portion to a signature defining one or more distinctive signal characteristic of the predefined facial muscle movement type.

In a first embodiment of the invention, the step of applying at least one facial muscle movement-detection algorithm to the bio-signals may include:

directly comparing bio-signals from one or more predetermined bio-signal detectors to the signature.

In another embodiment of the invention, the step of applying at least one facial muscle movement-detection algorithm to the bio-signals may include:

projecting bio-signals from a plurality of bio-signal detectors onto one or more predetermined component vectors; and

comparing the projections onto the one or more component vectors to that signature.

The predetermined component vectors may be determined from applying a first component analysis to historically collected bio-signals generated during facial muscle movements of the type corresponding to that first signature. The first component analysis applied to the historically collected bio-signals may be independent component analysis (ICA). Alternatively, the first component analysis applied to the historically collected bio-signals may be principal component analysis (PCA). In this embodiment, the method may further include the steps of:

applying a second component analysis to the detected bio-signals; and

using the results of the second component analysis to update the one or more predetermined component vectors during bio-signal detection.

The second component analysis may be principal component analysis (PCA).

In yet another embodiment of the invention, the step of applying at least one facial muscle movement-detection algorithm to the bio-signals may include:

applying a desired transform to the bio-signals; and

comparing the results of the desired transform to that signature.

The desired transform may be selected from any one or more of a Fourier transform, wavelet transform or other signal transformation method.

The step of applying at least one facial muscle movement-detection algorithm to the bio-signals may further include the step of:

separating the bio-signals resulting from the predefined type of facial muscle movement from one or more sources of noise in the bio-signals.

The sources of noise may include any one or more of electromagnetic interference (EMI), bio-signals not resulting from the predefined type of facial muscle movement and other muscle artefacts.

The step of applying one or more than one facial muscle movement-detection algorithm to the bio-signals may include comparing the sum or difference of bio-signals from one or more pairs of bio-signal detectors to that signature.

The step of applying one or more than one facial muscle movement-detection algorithm to the bio-signals may further include comparing bio-signals from each of the one or more pairs of bio-signal detectors to that signature.

The comparing step may include:

tracking a derivative of one or more that one of the bio-signals from each of the one or more pairs of bio-signal detectors and the sum or difference of bio-signals from the one or more pairs of bio-signal detectors.

The comparing step may further include:

comparing one or both of gradient and amplitude for one or more that one of the bio-signals from each of the one or more pairs of bio-signal detectors and the sum or difference of bio-signals from the one or more pairs of bio-signal detectors; and

determining when one or both of the gradient and amplitude respectively exceeds predetermined gradient and amplitude thresholds.

The comparing step may further include:

computing the correlation between bio-signals from each of the one or more pairs of bio-signal detectors; and

determining when the correlation exceeds a predetermined correlation threshold.

The step of applying one or more than one facial muscle movement-detection algorithm to the bio-signals may include comparing the power of bio-signals from one or more predetermined bio-signal detector to that signature.

The comparing step may include summing the power of bio-signals from one or more pairs of bio-signal detectors to that signature; and

determining whether the sum exceeds a predetermined threshold indicative of a first facial muscle movement type.

The comparing step may include computing the ratio of the power of bio-signals from a first group of bio-signal detectors to the power of bio-signals from a second group of bio-signal detectors; and

determining whether the ratio exceeds a predetermined threshold indicative of a second facial muscle movement type.

In one or more embodiments of the invention, the bio-signals may include any one or more of electroencephalograph (EEG) signals, electrooculograph (EOG) signals and electromyography (EMG) signals The method may further include the step of:

generating an output signal representative of the detected facial muscle movement type for input to an electronic entertainment application or other application.

Another aspect of the invention provides an apparatus for detecting and classifying facial muscle movements, including:

a processor and associated memory device for causing the processor to carry out the method described above.

Yet another aspect of the invention provides a computer program product, tangibly stored on machine readable medium, the product comprising instructions operable to cause a processor to carry out the method described above.

A further aspect of the invention provides a computer program product comprising instructions operable to cause a processor to carry out the method described above.

### FIGURES

These and other features, aspects and advantages of the present invention will become better understood with regard to the following description, appended claims, and accompanying figures which depict various views and embodiments of the device, and some of the steps in certain embodiments of the method of the present invention, where:

FIG. 1 is a schematic diagram of an apparatus for detecting and classifying facial muscle movements in accordance with the present invention;

FIG. 2 is a schematic diagram illustrating the positioning of scalp electrodes forming part of a head set used in the apparatus shown in FIG. 1;

FIG. 3 is a flow chart illustrating the broad functional steps performed by the apparatus in FIG. 1.

FIGS. 4 and 5 represent exemplary signals from selected electrodes shown in FIG. 2 during predefined facial movements;

FIG. 6 is a representation of signals from the scalp electrode shown in FIG. 2 during a number of facial muscle movements;

FIG. 7 is a flow chart illustrating the steps performed in the development of signatures defining distinctive signal characteristics of predefined facial muscle movement types used in the apparatus of FIG. 1 during the detection and classification of facial muscle movement;

FIG. 8 is a conceptual representation of the decomposition of signals from the sensors shown in FIG. 2 into predetermined components as performed by the apparatus of FIG. 1, in at least one mode of operation;

FIG. 9 is a representation of a signal from one of the sensors shown in FIG. 2 during a sequence of eye blinks;

FIG. 10 is a flow chart illustrating the steps performed by the apparatus of FIG. 1 both before and during bio-signal detection and classification in at least one mode of operation;

FIG. 11 is a schematic diagram showing an eye blink component vector present in the bio-signals captured from the sensors shown in FIG. 2 during an exemplary eye blink;

FIG. 12 is a flow chart of one exemplary algorithm for detecting and classifying facial muscle movements as eye blinks;

FIG. 13 shows a representation of a bio-signal detected from an exemplary sensor shown in FIG. 2 and subsequent analysis performed on that bio-signal;

FIG. 14 represents a flow chart of another exemplary algorithm for detecting and classifying facial muscle movements as eye blinks;

FIG. 15 is a flow chart of one exemplary algorithm for detecting and classifying facial muscle movements as smiles or clenches; and

FIG. 16 is a representation of signals from the sensors shown in FIG. 2 during a smile.

### DETAILED DECRIPTION

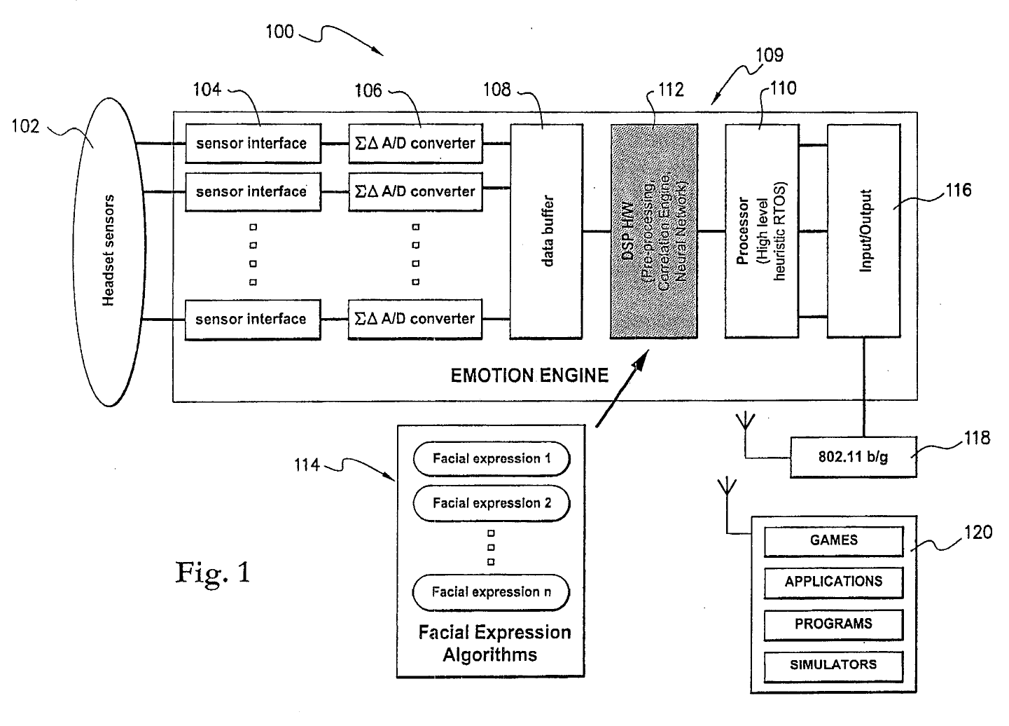

Turning now to FIG. 1, there is shown generally an apparatus 100 for detecting and classifying facial muscle movements. The apparatus 100 includes a headset 102 of bio-signal detectors capable of detecting various bio-signals from a subject, particularly electrical signals produced by the body, such as electroencephalograph (EEG) signals, electrooculograph (EOG) signals, electomyograph (EMG) signals or like signals. The apparatus 100 can also include bio-signal detectors capable of detecting other physiological signals, such as skin conductance. In the exemplary embodiment illustrated in the drawings, the headset 102 includes a series of scalp electrodes for capturing EEG signals from the user. The scalp electrodes may directly contact the scalp or alternately may be of the non-contact type that does not require direct placement on the scalp. Unlike systems that provide high-resolution 3-D brain scans, e.g., MRI or CAT scans, the headset is generally portable and non-constraining.

The electrical fluctuations detected over the scalp by the series of scalp sensors are attributed largely to brain tissue located at or near the skull. The source is the electrical activity of the cerebral cortex, a significant portion of which lies on the outer surface of the brain below the scalp. The scalp electrodes pick up electrical signals naturally produced by the brain and make it possible to observe electrical impulses across the surface of the brain. Although in this exemplary embodiment the headset 102 includes several scalp electrodes, in other embodiments only one or more scalp electrodes, e.g. sixteen electrodes, may be used in a headset.

Traditional EEG analysis has focused solely on these signals from the brain. The main applications have been explorative research in which different rhythms (alpha wave, beta wave, etc) have been identified, pathology detection in which onset of dementia or physical injury can be detected, and self improvement devices in which bio-feedback is used to aid in various forms of meditation. Traditional EEG analysis considers signals resulting from facial muscle movement such as eye blinks to be artefacts that mask the real EEG signal desired to be analysed. Various procedures and operations are performed to filter these artefacts out of the EEG signals selected.

The applicants have developed technology that enables the sensing and collecting of electrical signals from the scalp electrodes, and the application of signal processing techniques to analyze these signals in order to detect and classify human facial expressions such as blinking, winking, frowning, smiling, laughing, talking etc. The result of this analysis is able to be used by a variety of other applications, including but not being limited to electronic entertainment applications, computer programs and simulators.

Each of the signals detected by the headset 102 of electrodes is fed through a sensor interface 104, which can include an amplifier to boost signal strength and a filter to remove noise, and then digitized by an analogue-to-digital converter 106. Digitized samples of the signal captured by each of the scalp sensors are stored during operation of the apparatus 100 in a data buffer 108 for subsequent processing.

The apparatus 100 further includes a processing system 109 including a digital signal processor 112, a co-processing device 110 and associated memory device for storing a series of instructions (otherwise known as a computer program or computer control logic) to cause the processing system 109 to perform desired functional steps. Notably, the memory includes a series of instructions defining at least one algorithm 114 to be performed by the digital signal processor 112 for detecting and classifying a predetermined type of facial muscle movement. Upon detection of each predefined type of facial muscle movement, a corresponding control signal is transmitted in this exemplary embodiment to an input/output interface 116 for transmission via a wireless transmission device 118 to a platform 120 for use as a control input by electronic entertainment applications, programs, simulators or the like.

In one embodiment, the algorithms are implemented in software and the series of instructions is stored in the memory of the processing system, e.g., in the memory of the processing system 109. The series of instructions causes the processing system 109 to perform the functions of the invention as described herein. Prior to being loaded into the memory, the instructions can be tangibly embodied in a machine readable storage device, such as a computer disk or memory card, or in a propagated signal. In another embodiment, the algorithms are implemented primarily in hardware using, for example, hardware components such as application specific integrated circuits (ASICs). Implementation of the hardware state machine so as to perform the functions described herein will be apparent to persons skilled in the relevant art. In yet another embodiment, the algorithms are implemented using a combination of software and hardware.

Other implementations of the apparatus 100 are possible. Instead of a digital signal processor, an FPGA (field programmable gate array) could be used. Rather than a separate digital signal processor and co-processor, the processing functions could be performed by a single processor. The buffer 108 could be eliminated or replaced by a multiplexer (MUX), and the data stored directly in the memory of the processing system. A MUX could be placed before the A/D converter stage so that only a single A/D converter is needed. The connection between the apparatus 100 and the platform 120 can be wired rather than wireless.

Although the apparatus 100 is illustrated in FIG. 1 with all processing functions occurring in a single device that is external to the platform, other implementations are possible. For example, the apparatus can include a head set assembly that includes the head set, a MUX, A/D converter(s) before or after the MUX, a wireless transmission device, a battery for power supply, and a microcontroller to control battery use, send data from the MUX or A/D converter to the wireless chip, and the like. The apparatus can also include a separate processor unit that includes a wireless receiver to receive data from the headset assembly, and the processing system, e.g. the digital signal processor and the co-processor. The processor unit can be connected to the platform by a wired or wireless connection. As another example, the apparatus can include a head set assembly as described above, the platform can include a wireless receiver to receive data from the headset assembly, and a digital signal processor dedicated to detection of facial muscle movement can be integrated directly into the platform. As yet another example, the apparatus can include a head set assembly as described above, the platform can include a wireless receiver to receive data from the headset assembly, and the facial muscle movement detection algorithms are performed in the platform by the same processor, e.g., a general purpose digital processor, that executes the application, programs, simulators or the like.

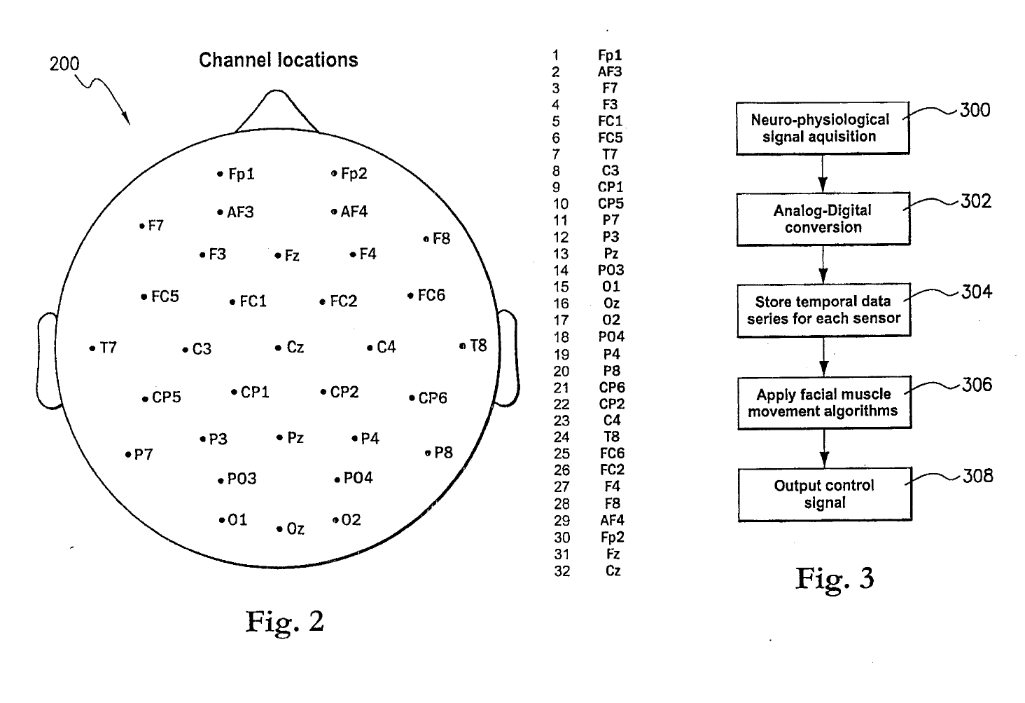

FIG. 2 illustrates one example of the positioning system 200 of the scalp electrodes forming part of the headset 102. The system 200 of electrode placement shown in FIG. 2 is referred to as the “10-20” system and is based on the relationship between the location of an electrode and the underlying area of cerebral cortex. Each point on the electrode placement system 200 indicates a possible scalp electrode position. Each site includes a letter to identify the lobe and a number or other letter to identify the hemisphere location. The letters F, T, C, P, O stand for Frontal, Temporal, Central, Parietal and Occipital. Even numbers refer to the right hemisphere and odd numbers refer to the left hemisphere. The letter Z refers to an electrode placed on the mid-line. The mid-line is a line along the scalp on the sagittal plane originating at the nasion and ending at the inion at the back of the head. The “10” and “20” refer to percentages of the mid-line division. The mid-line is divided into 7 positions, namely, Nasion, Fpz, Fz, Cz, Pz, Oz and Inion, and the angular intervals between adjacent positions are 10%, 20%, 20%, 20%, 20% and 10% of the mid-line length respectively.

The headset 102, including scalp electrodes positioned according to the system 200, is placed on the head of a subject in order to detect EEG signals. As seen in FIG. 3, at step 300, the EEG signals are captured by a neuro-physiological signal acquisition device and then converted into the digital domain at step 302 using the analogue to digital converters 106. A series of digitized signals from each of the sensors is then stored at step 304 in the data buffer 108. One or more facial muscle movement-detection algorithms are then applied at step 306 in order to detect and classify different facial muscle movements, including facial expressions or other muscle movements. Each of the algorithms generates a result representing the facial expression(s) of the subject. These results are then passed on to the output block 116 at step 308 where they can be used by a variety of applications.

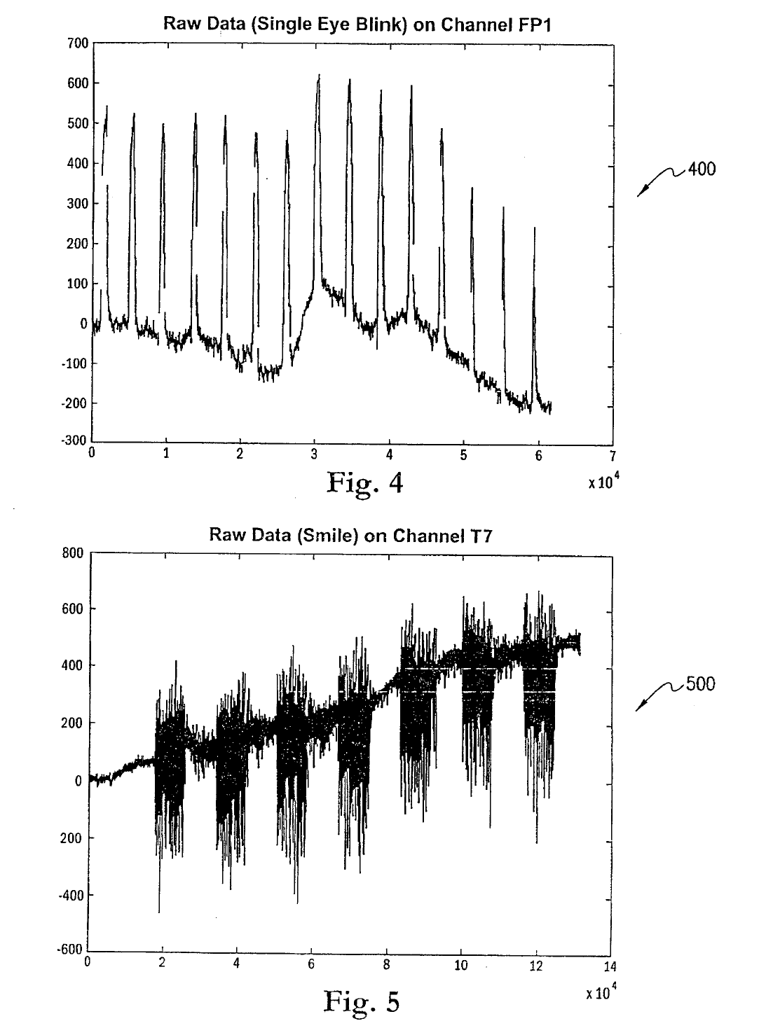

In traditional EEG research, many signals resulting from eye blinks and other facial muscle movements have been considered to be artefacts masking the real EEG signal required for analysis. FIG. 4 shows a representation 400 of a signal from the Fp1 or Fp2 electrode (as seen in the electrode positioning system 200 shown in FIG. 2) during a series of eye blinks. Similarly, FIG. 5 shows a representation 500 of a signal from the T7 or T8 electrode resulting from a series of smiles by a subject.

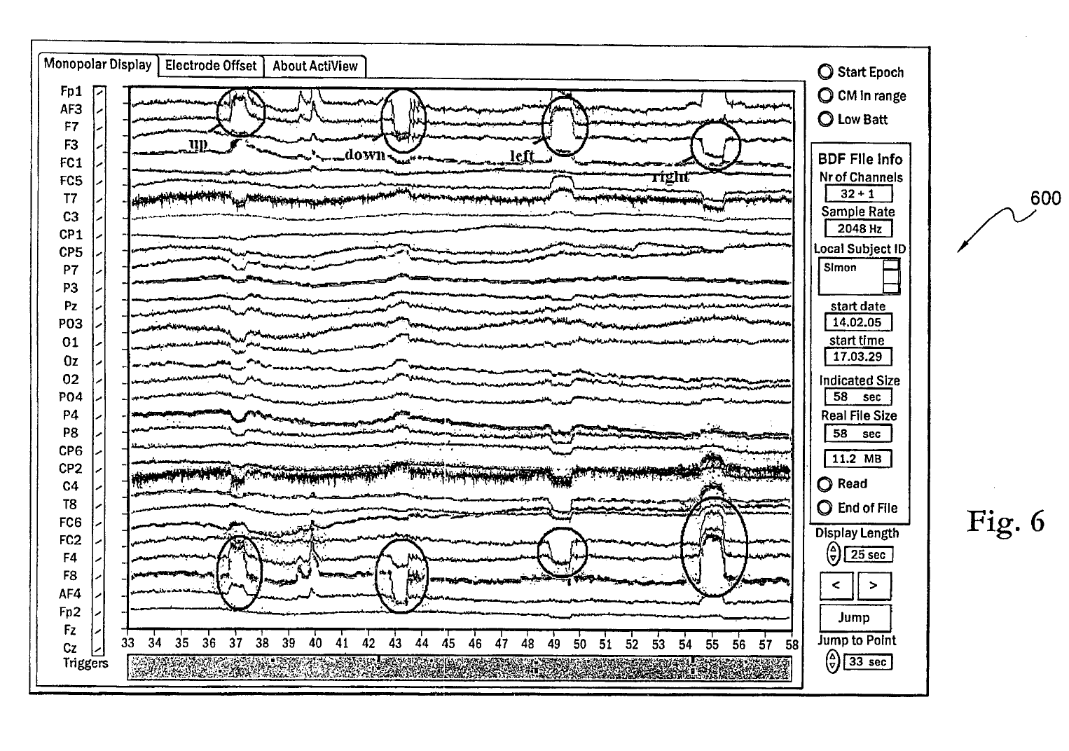

FIG. 6 shows a representation 600 of the signals from each of the electrodes in the headset 102 when various eye movements are performed by the subject. The impact of an up, down, left and right eye movement can be observed from the circled portions of signal representations. Rather than considering the impact upon the EEG signals resulting from facial muscle movements to be an artefact that pollutes the quality of the EEG signals, the apparatus 100 acts to isolate these perturbations and then apply one or more algorithms in order to classify the type of facial muscle movement responsible for producing the perturbations. The apparatus 100 applies at least one facial muscle movement-detection algorithm 114 to a portion of the bio-signals captured by the headset 102 affected by a predefined type of facial muscle movement in order to detect facial muscle movements of that predefined type. In order to do so, a mathematical signature defining one or more distinctive characteristics of the predefined facial muscle movement type is stored in the memory device 112. The relevant portion of the bio-signals affected by the predefined type of facial muscle movement is then compared to that mathematical signature.

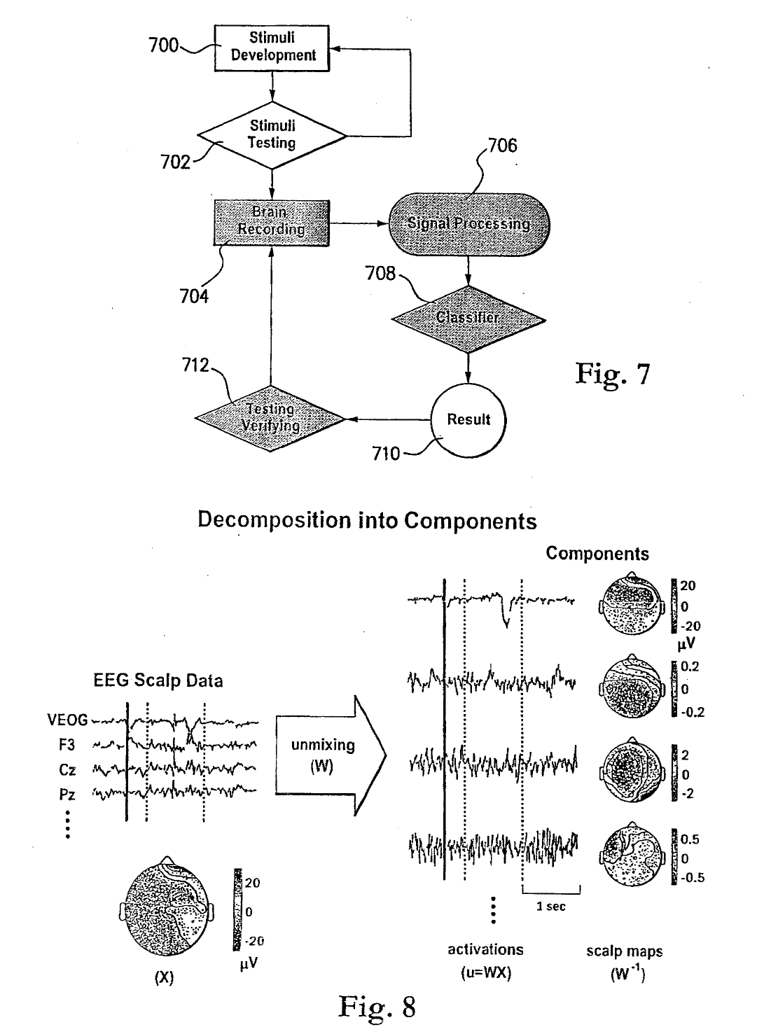

In order to generate the mathematical signature for each facial muscle movement, and as shown in FIG. 7, stimuli are developed at step 700 to elicit that particular facial expression. The stimuli are generally in the form of an audio visual presentation or a set of commands. The set of stimuli is tested at step 702 until a high degree of correlation between the developed stimuli and the resultant desired facial muscle movement is obtained. Once a set of effective stimuli is developed, EEG signal recordings are made at step 704 that contain many examples of the desired facial muscle movements. Ideally, these facial muscle movements should be as natural as possible.

Once the EEG signal recordings are collected, signal processing operations are then performed at step 706 in order to identify one or more distinctive signal characteristics of each predefined facial muscle movement type. Identification of these distinctive signal characteristics in each EEG signal recording enables classification of the facial muscle movement in a subject to be classified at step 708 and an output signal representative of the detected type of facial muscle movement to be output at step 710. Testing and verification of the output signal at step 712 enables a robust data set to be established.

In some embodiments, it may be necessary to develop a mathematical signature for each subject. In other embodiments of the invention, a generic mathematical signature can be developed for each type of facial muscle movement, e.g., using a limited number of subjects, and stored in the memory of the digital signal processor 112 without requiring the aforementioned steps to be carried out by each subject.

In one of the modes of operation, the portion of the bio-signals affected by a predefined type of facial muscle movement is predominantly found in signals from a limited number of scalp electrodes. For example, eye movement and blinking can be detected by using only two electrodes near the eyes, such as the Fp1 and Fp2 channels shown in FIG. 2. In this case, signals from those electrodes can be directly compared to the mathematical signatures defining the distinctive signal characteristics of the eye blink or other predefined facial muscle movement type.

It is also possible to combine the signals from one or more electrodes together, and then to compare that combined bio-signal to one or more signatures defining the distinctive signal characteristics of predefined facial muscle movement types. A weighting may be applied to each signal prior to the signal combining operation in order to improve the accuracy of the facial muscle movement detection and classification.

In other modes of operation, the apparatus 100 acts to decompose the scalp electrode signals into a series of components and then to compare the projection of the bio-signals from the scalp electrodes onto one or more predetermined component vectors with the mathematical signatures defining the signal characteristics of each type of facial muscle movement.

In this regard, independent component analysis (ICA) has been found to be useful for defining the characteristic forms of the potential function across the entire scalp. Independent component analysis maximizes the degree of statistical independence among outputs using a series of contrast functions. As seen in FIG. 8, in ICA, the rows of an input matrix X represent data samples from the bio-signals in the headset 102 recorded at different electrodes whereas the columns are measurements recorded at different time points. Independent component analysis finds an “unmixing” matrix W which decomposes or linearly unmixes the multi-channel scalp data into a sum of temporarily independent and specially fixed components. The rows of the output data matrix U=WX are time courses of activation of the ICA components. The columns of the inverse matrix, W-1, give the relative projection strength of each of the signals from the scalp electrodes onto respective component vectors. These scalp weights give the scalp topography of each component vector.

Another technique for the decomposition of the bio-signals into components is principal component analysis (PCA) which ensures that output components are uncorrelated. In various embodiments of the invention, either or both of independent component analysis and principal component analysis may be used in order to detect and classify facial muscle movements.

In other modes of operation, the apparatus 100 may act to apply a desired Fourier transform to the bio-signals from the scalp electrodes. The transform could alternatively be a wavelet transform or any other suitable signal transformation method. Combinations of one or more different signal transformation methods may also be used. Portions of the bio-signals affected by a predefined type of facial muscle movement may then be identified using a neural network.

Each of the above described techniques for detection and classification of the facial muscle movements may be incorporated into a facial muscle movement detection algorithm stored in the memory of and performed by the digital signal processor 112. Once a particular facial muscle movement detection algorithm has been fully developed, the algorithm may be implemented as a piece of real-time software program or transferred into a digital signal processing or other suitable environment.

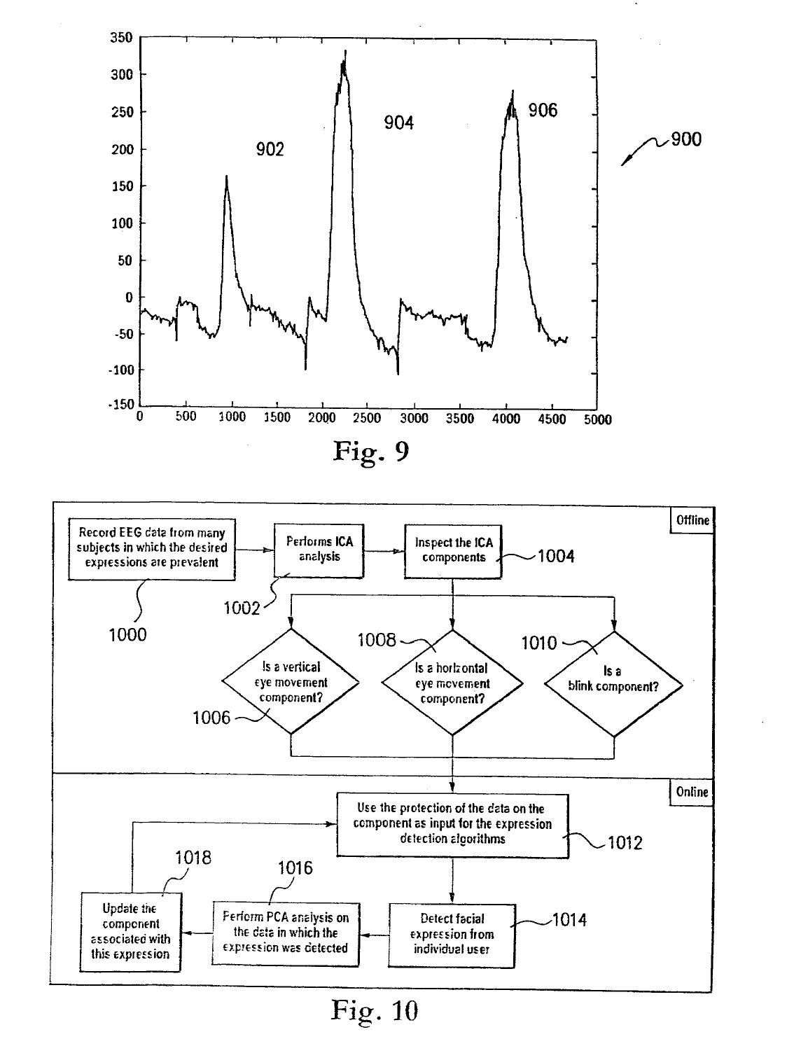

As an example of the type of facial muscle movement that can be detected and classified by the apparatus 100, a facial expression algorithm for the detection of an eye blink will now be described. It is to be understood that the general principles described in relation to the algorithm are also applicable to the detection and classification of other types of facial muscle movement, such as winks and eyeball motions. Eye blinks are present in all anterior electrodes but feature most prominently in the two frontal channels Fp1 and Fp2. FIG. 9 is a representation 900 of the bio-signal recorded at the scalp electrode Fp1 during 3 typical eye blinks. It can be seen from signal portions 902, 904 and 906 of the bio-signal from the frontal channel Fp1 that each of the 3 eye blinks has a significant effect on the bio-signal. In this example, the projections of the bio-signals from the frontal electrodes Fp1 and Fp2 on predetermined component vectors are used to detect and classify the perturbation in the bio-signals as an eye blink.

In one embodiment of the invention, the predetermined component vectors are identified from historically collected data from a number of subjects and/or across a number of different sessions. As shown in FIG. 10, the EEG data from a number of different subjects and/or across a number of different sessions are recorded at step 1000 when the desired facial muscle movements are being generated by the subjects. At step 1002, independent component analysis is performed on the recorded EEG data and the component vectors onto which are projected the perturbations in the EEG signals resulting from the relevant facial muscle movement are determined at step 1004. The relevant component vectors to be used in subsequent data recording and analysis are then recorded in the storage device 112 by facial muscle movement type. In this case, three exemplary types of facial muscle movement are able to be classified, namely vertical eye movement at step 1006, horizontal eye movement at step 1008 and an eye blink at step 1010.

Independent component analysis is a computationally time consuming activity and in many instances is inappropriate in some application, such as real-time use. Whilst independent component analysis may be used to generate average component vectors for use in the detection and classification of various types of facial muscle movements, the balance of signals across different electrodes vary slightly across different sessions and users.

Accordingly, the average component vectors defined using independent component analysis of historically gathered data may not be optimal during real-time data detection and classification. During real-time operation of the apparatus 100, principal component analysis can be performed on the real-time data and the resulting component vector can be used to update the component vector generated by independent component analysis throughout each session. In this way, the resulting facial muscle movement-detection algorithms can be made robust against electrodes shifting and variances in the strengths of electrode contact.

As can be seen at step 1012, the projection of the historically collected data on the vector component is initially used as a reference in the facial muscle movement detection algorithms 114. However, as data is collected and stored in the data buffer 108 at step 1014, principal component analysis is carried out at step 1016 on the stored data, and the results of the analysis generated at step 1018 are then used to update the component vectors developed during offline independent component analysis.

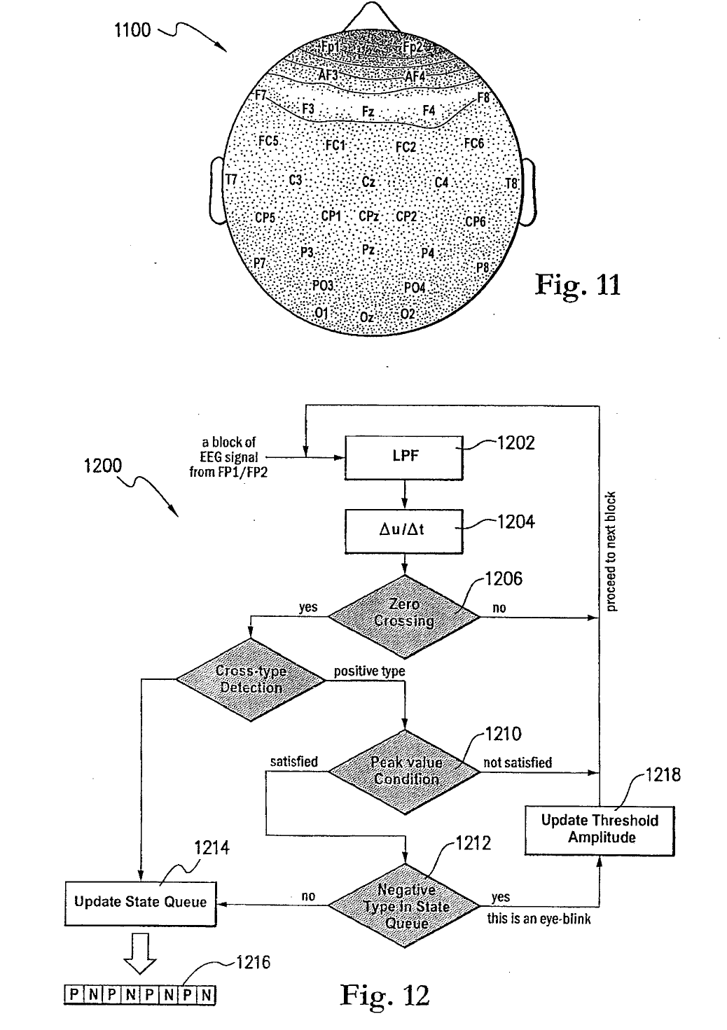

As has been previously described, component vectors can be used in order that a correct weighting is applied to the contribution from the signals of each relevant electrode. An example of an eye-blink component vector is shown in the vector diagram 1100 in FIG. 11. From this diagram it can be seen that the largest contribution to the component is indeed from the two frontal electrodes Fp1 and Fp2. However, it is also apparent that the eye blink is not symmetric. In this case, the potential around the electrode Fp2 is larger than that of the electrode Fp1. The difference may be due to a number of causes, for example, muscle asymmetry, the electrodes not being symmetrically located on the head of a subject or a difference in the electrical impedance contact with the scalp. This diagram illustrates the desirability of optimizing the component vectors during each session, for example by applying the steps illustrated in FIG. 10.

FIG. 12 shows one example of a facial muscle movement-detection algorithm 1200 used to detect an eye blink. The algorithm 1200 may be applied to the activations of component vectors or alternatively may be applied to signals from individual scalp electrodes or from combinations of signals from more than one scalp electrode. In one embodiment, the projection of the EEG signals onto the component vector associated with an eye blink is initially passed through a low pass filter at step 1202. A first order derivative operation is then performed on the signal at step 1204. In short, the first order derivative of a function ƒ with respect to an infinitesimal change in x is defined as

$${f}^{1} ( x ) ≡ {lim}_{h \to 0} \frac{f ( x + h ) - f ( x )}{h} .$$

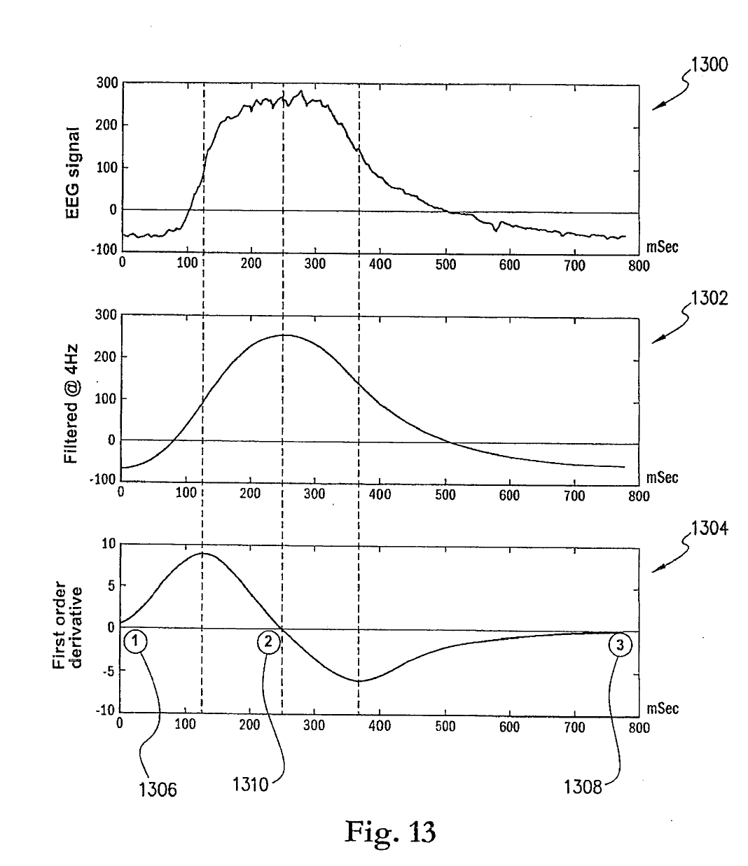

The result of this process for a single eye blink is shown in FIG. 13. The original component vector is referenced 1300, whereas the signal resulting from the low pass filtering, and from the first order derivative operation are referenced 1302 and 1304 respectively.

Of particular interest are zero-crossing points in the first order derivative signal, which fall into two categories: positive zero-crossing point and negative zero-crossing point. The sign (namely either positive or negative) of the zero-crossing points indicates whether the signal increases or decreases after crossing the axis. For each eye blink, there are two positive zero-crossing points, respectively referenced 1306 and 1308 on FIG. 13. These positive zero-crossing points define boundary conditions of an eye blink. A negative zero-crossing point 1310 defines the peak of the eye blink. Accordingly, the algorithm 1200 determines at step 1206 whether a zero-crossing point occurs in the digitized data stored in data buffer 108. If this is the case, a determination is made at step 1208 if the crossing type is a positive or a negative zero-crossing. If a negative crossing is detected, the peak amplitude of the corresponding signal is checked at step 1210 to verify whether this transitory rise in signal amplitude is from a real eye blink. If a positive zero-crossing point is detected, the algorithm stores this information into state queue at step 1214 in cases where there is no preceding negative zero-crossing point whose corresponding signal amplitude satisfying the peak value condition determined at step 1212 stored in the queue. If there is a preceding negative zero-crossing point stored in the state queue, an assertion that there is an eye blink is made at step 1212. The algorithm resets if the found negative zero-crossing point does not satisfy peak value condition or an eye blink detection assertion is made.

Accordingly, once the zero-crossing points are identified, the algorithm verifies whether there exists a negative zero-crossing point sandwiched between the two positive zero-crossing points, and the eye blink peak passes amplitude threshold. A default value of the amplitude threshold is initially made, but to increase the accuracy of the algorithm, the threshold amplitude is optionally adjusted at step 1218 based upon the strength of an individual's eye blink peaks.

In this example, the eye blink “signature” defines the distinctive signal characteristics representative of an eye blink, namely a negative zero crossing sandwiched between two positive zero crossings in the first order derivative of the filtered signal, and a signal amplitude greater than a predetermined threshold in the filtered signal. The signature is optionally updated by changing the threshold forming part of the distinctive signal characteristics of the signature during facial muscle movement detection and classification. In other embodiments, the digital signature may define other amplitudes or signal characteristics that exceed one or more predetermined thresholds. The signature may be updated during facial muscle movement detection and classification by changing one or more of those thresholds. More generally, any one or more distinctive signal characteristics of a predetermined facial muscle movement type that form part of a digital signature can be updated during the course of facial muscle movement detection and classification in order to improve the viability and accuracy of the facial muscle movement detection algorithms implemented by the apparatus 100.

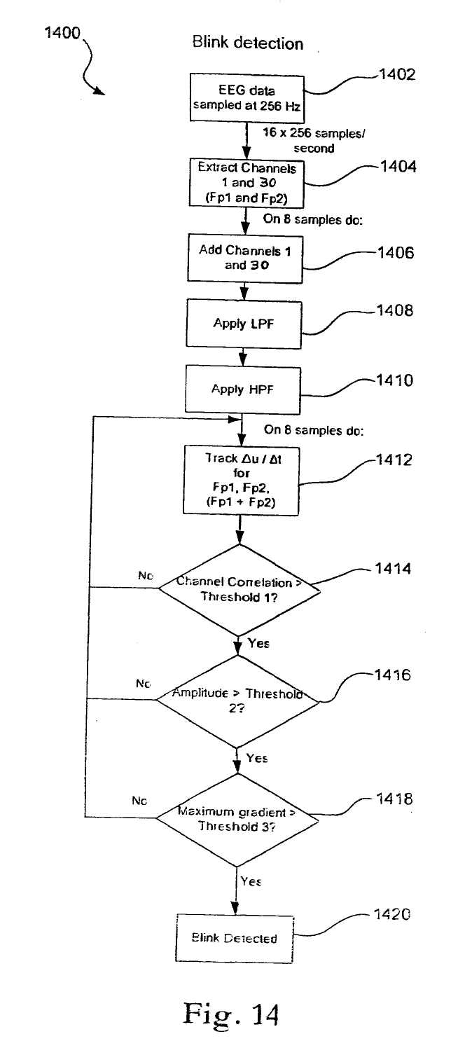

The specific channels used to detect and classify various facial expressions may differ according to the particular facial expression in question. In addition to using signals from individual channels or activations of component vectors, the sum or difference of channel pairs may be used in facial muscle movement detection algorithms. FIG. 14 shows another example of a facial muscle movement detection algorithm 1400 used to detect an eye blink. The algorithm 1400 involves examining the correlation of signals between channel pairs, as well as the amplitude and gradient of bio-signals from each of the pair of channels Fp1 and Fp2, as well as the sum of bio-signals from that pair of channels. In this exemplary algorithm, at step 1402, bio-signals from the sensors shown in FIG. 2 are sampled at a rate of 256 Hz resulting in 256 samples per second being generated for each of the 32 channels. At step 1404, channels 1 and 30 corresponding respectively to bio-signals from the sensors Fp1 and Fp2 are extracted for a data window corresponding to one over 32 of a second containing 8 samples. At step 1406, samples from channels 1 and 30 are summed.

At step 1408, a third order infinite impulse response (IIR) low pass filter is applied at 10 Hz, whilst at step 1410 a first order IIR high pass filter is applied at 0.125 Hz.

At step 1412, a first order derivative operation is performed on the sum of bio-signals from channels Fp1 and Fp2. Similar to the aforementioned algorithm 1200, an eye blink peak is tracked by the negative zero-crossing point of the first order derivative. The rise and the fall of an eye blink signal peak are bounded by positive zero-crossing points preceding and following this negative zero-crossing point of the first order derivative, respectively. An assessment is made as to whether the correlation between the filtered signals of channels Fp1 and Fp2 for the window of data bounded by a positive zero-crossing and a negative zero-crossing for the rise of a peak (and vice versa for the fall of a peak) exceeds a first predetermined threshold at step 1414, whether the lesser amplitude of the rise or the fall of an eye blink peak signal from either of the individual channels Fp1 and Fp2 exceeds a second predetermined threshold at step 1416, and whether the maximum gradient determined from the peak or trough of the first order derivative of the summed signals from channels Fp1 and Fp2 exceeds a third predetermined threshold at step 1418. If these three values are above their respective thresholds in all cases, then an eye opening or eye closing event is detected at step 1420 (dependent on whether the maximum gradient is positive or negative).

Similar algorithms can be used to identify winks, eyeball motions or other related facial muscle expressions. Other algorithms may use different combinations of signal correlation, amplitude displacement and signal gradient measurement, as well as assessing the sum or difference of bio-signals from one or more different channel pairs to those used in the algorithm illustrated in FIG. 14.

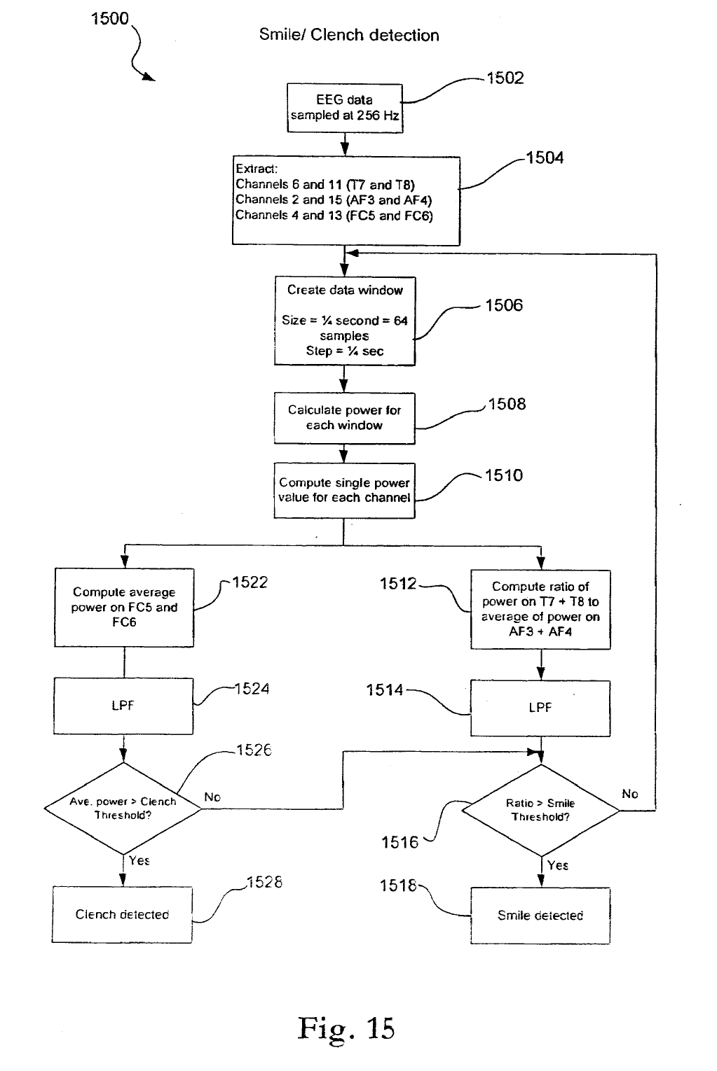

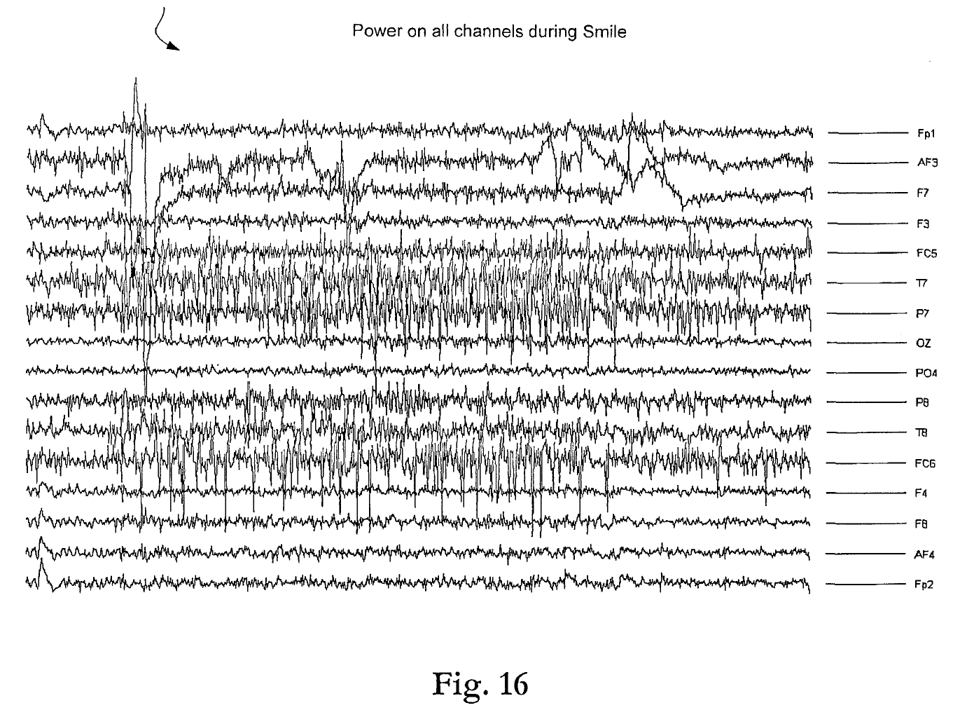

Other algorithms used to detect and classify facial muscle movements rely upon the determination of signal power upon particular channels, the sum of signal power on one or more pairs of signal channels, the difference of signal power between one or more pairs of channels and/or the ratio of the signal power on one or more channels or one or more channel pairs to the power of signals on one or more other channels or one or more other channel pairs. By using the exemplary algorithm 1500 shown in FIG. 15, either a smile or clench can be detected. This exemplary algorithm determines a metric that is calculated by using the signal power on particular channels, signal power on the sum of bio-signals on particular channels and ratios of the sums of signal power on signal channel pairs. At step 1502, the bio-signals from each of the detectors shown in FIG. 2 are sampled at 256 Hz. At step 1504, data from several channel pairs is extracted, namely data from channel 6 and 11 corresponding to detectors T7 and T8, channels 2 and 15 corresponding to detectors AF3 and AF4, channels 4 and 13 corresponding to detectors FC5 and FC6 and channels 3 and 14 corresponding to detectors F7 and F8.

At step 1506, a data window is created for each of these channels in which 64 consecutive samples (corresponding to a quarter of a second) are considered. At step 1508, the power of the signal represented by the 64 consecutive samples in the data window for each channel is calculated, and a single power value computed for each channel at step 1510.

In order to determine whether a smile is present in the EEG data sample in step 1502, at step 1512 the ratio of the power on channel pair T7 and T8 to the average of the power on channel pair AF3 and AF4 and channel pair FC5 and FC6 is computed. At step 1514, a low pass filter is applied to the computation carried out at step 1512. At step 1516, a determination is made as to whether the ratio computed at step 1512 exceeds a predetermined threshold indicative of a smile. If this is the case, then a smile is detected at step 1518, or otherwise step 1506 is performed again with the next 64 data samples for each of the channels extracted at step 1504.

Whilst step 1512 is being computed in order to determine whether a smile is present in the bio-signal data, at step 1522 the average power on channel pair FC5 and FC6 is being computed in order to determine whether a clench is present in the bio-signals. If it is determined at step 1526 that this computed average power exceeds a predetermined threshold indicative of a clench, then a clench is detected at step 1528. If the average power computed in step 1522 does not exceed the predetermined threshold indicative of a clench at step 1526, then the power ratio computed at step 1512 is compared to the threshold indicative of a smile in order to determine whether a smile is present in the bio-signals.

In order to improve the efficiency of algorithms relying upon signal power detection, a signal power profile can be created using signal power on all channels to create a signal power profile as a 32 channel vector. There are several ways of creating a signal power profile for an expression. For example, a combination of principle component analysis, simple statistics such as mean, median and manual inspection can be used to create the signal power profile. The profile can then be normalized to a unit vector for scaling simplicity. The dot product of the signal power profile and the normalized signal power on all channels can then be used as the signal to identify when a particular facial expression occurs.

In the algorithm described in relation to FIG. 15, signal power on a channel is calculated by taking the first order derivative of the channel signal, which is sometimes firstly passed through a high pass filter. The absolute value of the first order derivative of a channel signal is then taken, and some fraction of the lowest and highest values is discarded. These values may typically be ⅜ of the lowest and the ⅛ of the highest values. The mean of the non-discarded portion is then calculated. The data window used to calculate the signal power is normally in the order of ¼ of a second.

Alternative methods of calculating signal power can be used in other embodiments of the invention. These methods may be based upon signal power present in particular frequency bands on channels, or the ratio of power in two different frequency bands or two different channels. These could be different channels on the same band, different bands on the same channel or different bands on different channels.

The channel correlation performed in relation to the algorithm shown in FIG. 14 can be calculated according to the expression sum (xy)/sqrt(sum(x²)+sum(y²)). In some embodiments of the invention however, an alternative measure of correlation is carried out: sum(xy)/sqrt(sum(max(x,y)²)). In both of the two preceding expressions, the terms x and y correspond to the two signals being calculated. This latter correlation method has the advantage of reducing the value in two signals that have similar profiles, but one is very much larger than the other.

The facial expression detection algorithms described above can have multiple threshold values associated therewith. These threshold values may not be intuitive to adjust and it is therefore useful to be able to translate these thresholds into one or more intuitive “sensitivity” parameters.

For example, the effect that an eye blink has on the EEG signal from any one or more of the detectors shown in FIG. 2 can be simply understood as concurrent upward deflections on a number of frontal channels. These deflections may be characterized by the minimum height of the deflection, the minimum gradients of the deflection and the minimum correlations of the signals on different channels. How these three thresholds are combined is not necessarily straightforward to the non-expert user. In order to convert the multiple thresholds to a single sensitivity parameter, it may be desirable to evaluate a reasonable range for each threshold. Each threshold may then be interpolated, either linearly or otherwise, between the minimum and maximum values of the reasonable range. Accordingly, if the minimum threshold value for an individual parameter is thresh_min and the maximum is thresh_max. Given a sensitivity parameter of s that varies on a range of 0 to 1 and using linear interpolation, the threshold value for that parameter would be:
threshold=thresh_max+(thresh_min−thresh_max)*s
Additionally the sensitivity threshold may be inferred by any of the individual thresholds based on:
s=(threshold−thresh_max)/(thresh_min−thresh_max)
Due to the variance in musculature of different people, the expressions detected via noise profiling may have very different values. Automatic calibration of these algorithms can be performed to cater for this variability. Calibration can be performed by recording a “neutral” state, defined as anything but the expressions that are being calibrated. Noise values are calculated for this period, the values obtained are sorted and the lower 50% of the values are discarded. The average of the remaining values is then used as a baseline above which we can assume that there is some small amount of expression present.

Calibration of the expression's maximum threshold can then be done by using a multiple of the lower threshold, for example 2 or 3 times the lower threshold. Alternatively the subject can be asked to perform the expression, and the values obtained during this period can be used to determine upper end of the facial expression range. Care should be taken as the perturbations caused by forced expressions may not be as large as naturally occurring expressions, so the maximum value found during such a calibration should be set to 50% of the range.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference in their entirety.

1. A method of detecting and classifying facial muscle movements, including the steps of:
a) receiving bio-signals from one or more than one bio-signal detector; and
b) applying one or more than one facial muscle movement-detection algorithm to a portion of the bio-signals affected by a predefined type of facial muscle movement in order to detect the facial muscle movements of the predefined type.

2. The method according to claim 1, wherein the step of applying one or more than one facial muscle movement-detection algorithm to the bio-signals includes comparing the bio-signal portion to a signature defining one or more than one distinctive signal characteristics of the predefined facial muscle movement type.

3. The method according to claim 2, wherein the step of applying one or more than one facial muscle movement-detection algorithm to the bio-signals includes directly comparing bio-signals from one or more than one predetermined bio-signal detectors to that signature.

4. The method according to claim 2, wherein the step of applying one or more than one facial muscle movement-detection algorithm to the bio-signals includes:
a) projecting bio-signals from the plurality of bio-signal detectors on one or more than one predetermined component vectors; and
b) comparing the projection of the bio-signals onto one or more than one component vectors to that signature.

5. The method according to claim 4, further including applying a desired transform to the projected bio-signal after the projection of the bio-signals from the plurality of detectors on one or more than one component vectors, and before the projected bio-signal is compared to that signature.

6. The method according to claim 4, wherein the predetermined component vectors are determined by applying a first component analysis to historically collected bio-signals generated during facial muscle movement types of the type corresponding to that signature.

7. The method according to claim 6, wherein the first component analysis applied to the historically collected bio-signals is independent component analysis (ICA).

8. The method according to claim 6, wherein the first component analysis applied to the historically collected bio-signals is principal component analysis (PCA).

9. The method according to claim 4, wherein the one or more than one component vectors are updated during facial muscle movement-detection and classification.

10. The method according to claim 2, further including updating the signature during the course of facial muscle movement-detection and classification.

11. The method according to claim 10, wherein the signature is updated by changing thresholds forming at least part of the distinctive signal characteristics of the signature.

12. The method according to claim 2, wherein the step of applying one or more than one facial muscle movement-detection algorithm to the bio-signals includes:
a) applying a desired transform to the bio-signals; and
b) comparing the results of the desired transform to that signature.

13. The method according to claims 12, wherein the transform is one or more than one transform selected from the group consisting of a Fourier transform and a wavelet transform.

14. A method according to claim 4 further including:
a) applying a second component analysis to the detected bio-signals; and
b) using the results of the second component analysis to update the one or more than one predetermined component vectors during bio-signal detection.

15. The method according to claim 14, wherein the second component analysis is principal component analysis (PCA).

16. The method according to claim 1, wherein the step of applying one or more than one facial muscle movement-detection algorithm to the bio-signals includes separating the bio-signals resulting from the predefined type of facial muscle movement from one or more than one sources of noise in the bio-signals.

17. The method according to claim 16, wherein the sources of noise comprise one or more than one source selected from the group consisting of electromagnetic interference (EMI), and bio-signals not resulting from the predefined type of facial muscle movement.

18. The method according to claim 2, wherein the step of applying one or more than one facial muscle movement-detection algorithm to the bio-signals includes comparing the sum or difference of bio-signals from one or more pairs of bio-signal detectors to that signature.

19. The method of claim 18, wherein the step of applying one or more than one facial muscle movement-detection algorithm to the bio-signals further includes comparing bio-signals from each of the one or more pairs of bio-signal detectors to that signature.

20. The method of claim 19, wherein the comparing step includes:
tracking a derivative of one or more than one of the bio-signals from each of the one or more pairs of bio-signal detectors and the sum or difference of bio-signals from the one or more pairs of bio-signal detectors.

21. The method of claim 20, wherein the comparing step further includes:
comparing one or both of gradient and amplitude for one or more than one of the bio-signals from each of the one or more pairs of bio-signal detectors and the sum or difference of bio-signals from the one or more pairs of bio-signal detectors; and
determining when one or both of the gradient and amplitude respectively exceeds predetermined gradient and amplitude thresholds.

22. The method of either of claims 20 or 21, wherein the comparing step further includes:
computing the correlation between bio-signals from each of the one or more pairs of bio-signal detectors; and
determining when the correlation exceeds a predetermined correlation threshold.

23. The method according to claim 2, wherein the step of applying one or more than one facial muscle movement-detection algorithm to the bio-signals includes comparing the power of bio-signals from one or more predetermined bio-signal detector to that signature.

24. The method according to claim 23, wherein the comparing step includes summing the power of bio-signals from one or more pairs of bio-signal detectors to that signature; and
determining whether the sum exceeds a predetermined threshold indicative of a first facial muscle movement type.

25. The method according to claim 23, wherein the comparing step includes computing the ratio of the power of bio-signals from a first group of bio-signal detectors to the power of bio-signals from a second group of bio-signal detectors; and
determining whether the ratio exceeds a predetermined threshold indicative of a second facial muscle movement type.

26. The method according to claim 1, wherein the bio-signals include electroencephalograph (EEG) signals.

27. The method according to claim 1, further including generating an output signal representative of the detected facial muscle movement type.

28. An apparatus for detecting and classifying facial muscle movements, including:
a processor and associated memory device for causing the processor to carry out a method according to claim 1.

29. A computer program product, tangibly stored on machine readable medium, the product comprising instructions operable to cause a processor to carry out a method according to claim 1.
